(19) United States
(12) Patent Application Publication
(10) Pub. No.: US 2014/0148878 A1
(43) Pub. Date: May 29, 2014

(54) NON-SURGICAL METHOD FOR TIGHTENING BOTH EXTERNAL PUBIC AND INTERNAL VAGINAL TISSUES IN A SINGLE PROCEDURE

(71) Applicant: Khalilullah A. Khatri, Lexington, MA (US)

(72) Inventor: Khalilullah A. Khatri, Lexington, MA (US)

(21) Appl. No.: 14/088,347

(22) Filed: Nov. 23, 2013

Related U.S. Application Data
(60) Provisional application No. 61/731,049, filed on Nov. 29, 2012.

(51) Int. Cl. A61F 7/00; A61F 7/12; A61N 5/06

(52) U.S. Cl. CPC A61F 7/007; A61N 5/0603; A61N 5/0616; A61F 7/12
USPC 607/89; 607/108; 607/100

(57) ABSTRACT

A non-surgical method for improving both the external pubic appearance and the internal vaginal tightness of a female patient in a single procedure includes providing a cosmetic skin-tightening device having a wand having a tip that applies heating energy to skin collagen, where the wand is shaped such that the tip can be inserted into the patient's vagina. During the procedure, the wand is alternately passed over the external pubic skin and inserted into the patient's vagina, thereby tightening both the pubic and vaginal regions during a single procedure while preventing overheating of any one area. The heat energy can be conductively transmitted heat or a radiated energy such as infra-red, laser, or radio frequency energy. A disposable sleeve can be applied over the wand to facilitate cleaning and sterilization after use. A vaginal lubricant and/or ultrasound gel can be applied.

### RELATED APPLICATIONS

This application claims the benefit of U.S. Provisional Application No. 61/731,049, filed Nov. 29, 2012, which is herein incorporated by reference in its entirety for all purposes.

### FIELD OF THE INVENTION

The invention relates to cosmetic procedures, and more particularly, to procedures for shrinking and tightening tissue structures in the pubic and vaginal areas.

### BACKGROUND OF THE INVENTION

No one can avoid the process of aging. However, in a society where having a youthful appearance is prized, many people have a strong desire to maintain and rejuvenate their appearance as much as they possibly can. To some degree, this can be achieved by taking good care of the body, through such measures as maintaining a sensible diet, engaging in regular exercise, using sun screen when outdoors, and using skin creams to prevent skin dryness. However, these steps do not retard the natural sagging and loosening of skin that takes place over time in virtually all regions of the body, and which is a major contributor to the aging of one's appearance.

Surgery can be used to cosmetically reduce the appearance of aging by removing excess skin and joining the remaining skin back together, so that the skin is tightened to a more youthful state. However, surgery is expensive, risky, and is typically followed by a lengthy and painful recovery period.

Another approach is to use heat, chemicals, abrasives, lasers, and/or radio frequency (RF) irradiation to heat, destroy, and/or remove collagen in layers of the skin, thereby triggering growth of new collagen (neo-collagenesis) that results in skin tightening through a process similar to scar tissue growth, but without any visible appearance of scarring.

These surgical and non-surgical skin-tightening approaches are widely used on areas of the body that are normally exposed, especially the face and neck. However, the desire for a more youthful appearance is not confined only to areas of the body that are normally exposed. Loose or hanging skin and muscles in other areas of the body can be observable beneath certain clothing, and can therefore limit fashion choices. Also, concerns about presenting a youthful appearance can be especially high during sexual intimacy, where physical attraction plays a vital role, and where parts of the body that are normally hidden are exposed to view by a partner. In particular, for many women the attractiveness of their pubic area to a sexual partner can be a source of great concern and anxiety.

Female exterior genitalia typically undergo changes that are similar to the changes that occur in the rest of the body. With age and childbirth, as well as weight changes, the labia and surrounding tissue change in shape, size and tone. Sagging of the tissues can lead to an enlarged, bulging, and sagging appearance of the overall pubic area, including the labia and vulva, and can also lead to increased separation of the labia. Other problems can include larger than normal labia size (which can lead to personal hygiene issues, discomfort during intercourse, discomfort during exercise, and recurring infections), asymmetry of the labia, and hyperpigmentation.

Many women fear that these changes will cause them to have an aged and therefore less attractive appearance to their sexual partner. Also, a woman may feel a need as she ages to avoid certain clothing, for example tight pants, which might otherwise be sexually attractive, but which might cause her enlarged and/or sagging pubic area to be apparent to others.

Similar changes also occur within the vagina. Due to the aging process and/or for women who've experienced multiple childbirths, the vagina may sag and/or become stretched, and can lose its tone and strength. The result can often be loose, weak, vaginal wall/canal and muscles. Many women fear that these changes will decrease the pleasure of sex, both by them and by their partners.

Generally, concerns about the external appearance of the pubic area and about the internal changes within the vagina are viewed as separate problems, and are addressed by separate approaches and procedures.

Regarding the external appearance of the pubic area, a variety of surgical procedures are commonly used to tighten skin and reverse some of the visible aging effects and other aesthetic issues. For example, in a “Laser Reduction Labiaplasty,” a laser is used for excision of excess labial tissue. In some cases, surgical excision is combined with laser irradiation to further enhance the tightening effect while also providing a smoother texture. Also, liposuction is sometimes used to shrink areas such as the Mons Pubis region.

Regarding the interior changes within the vagina, a surgical procedure typically referred to as vaginoplasty can be used to improve internal tightness. In a typical vaginoplasty, the stretched muscle at the back of the vagina is joined together and shortened with dissolvable stitches, and the unwanted tissue is removed. This tightens the vaginal canal and surrounding soft tissues, by reducing excess and loose vaginal tissue.

However, while many women may strongly desire the benefits of these various procedures, the procedures themselves can be impractical and/or highly undesirable. Surgery is expensive, risky, and typically requires a lengthy and painful recovery period. Generally, a different, specialized apparatus is required for each type of surgical procedure. In addition, each surgical procedure must be performed by a surgeon, if not a full surgical team. Hence, for many patients it can be prohibitively expensive to have even one procedure, let alone a program of several procedures so as to address both external pubic and internal vaginal concerns. Also, as mentioned above, surgery can be painful, and can require a long recovery time.

What is needed, therefore, is a non-surgical method whereby a single procedure is applied by a single, appropriately trained technician using a single cosmetic apparatus to improve both the external pubic appearance and the internal vaginal tightness of a woman's body, thereby providing a more youthful appearance and heightened sexual pleasure without excessive costs, pain, or recovery time.

### SUMMARY OF THE INVENTION

The present invention is a non-surgical method whereby a single procedure is applied by a single, appropriately trained technician using a single cosmetic apparatus to improve both the external pubic appearance and the internal vaginal tightness of a female patient's body, thereby providing a more youthful appearance and heightened sexual pleasure without excessive costs, pain, or recovery time. The method employs a device that is at least similar to devices typically used to tighten external skin in regions of the body such as the abdomen, thighs, love handles, face, and neck that are normally visible. However, in the present invention the device is used in a novel manner that was not foreseen by previous device manufacturers.

Specifically, a device for heating collagen in the dermal layer of the skin is used, whereby the device includes a wand attached to a control unit by an umbilical cable. When placed next to a patient's skin, the wand is configured to cause heating of collagen in the skin, which causes the collagen to contract and thicken, and also triggers the creation of new collagen, which leads to tightening of the skin. In various embodiments, the heating is caused by direct thermal transfer, or by application of a radiated energy such as infrared, laser, or radio frequency energy emitted by the wand.

According to the present invention, the device includes a wand that has a size and shape allowing it to be inserted within the vagina. In some embodiments, the wand is a standard accessory typically used for the face, neck, and/or other normally visible regions of the body. In other embodiments, the wand is provided specifically for the purpose of applying the method of the present invention. Because the wand can be inserted into the vagina, it can be used to heat and tighten internal tissue within the vaginal canal, as well as heating and tightening skin in the external pubic area. During the procedure, the wand is alternately passed over the pubic area and then inserted and moved about within the vagina. By shifting the wand frequently between different areas, excessive overheating and discomfort in any one area is avoided. Typically, the patient feels a warming sensation, but does not experience any undue discomfort, so that anesthesia is not typically required.

Accordingly, during a single non-surgical procedure both the external skin of the pubic area and the internal tissue within the vagina are heated and/or irradiated, and are thereby tightened. The method can be repeated after a minimal recovery period, in some embodiments up to six times or more, so as to provide a greater net effect. In various embodiments, the wand is covered during the procedure by a cleanable or disposable cover, so as to facilitate cleaning and sterilization of the wand after use. In various embodiments, the cover is specially designed for use with the device, while in other embodiments an item designed for a different purpose, such as a finger of a surgical glove, is used as a cover. In some of these embodiments, the tip of the cover or glove finger is cut off so as to avoid interfering with the heating at the wand tip.

The present invention is a method applicable to a female patient for tightening tissue in both the external pubic area and the internal vaginal area during a single treatment session using a single device. The method makes use of a cosmetic device that is able to emit energy from a wand that heats collagen in skin, the wand having an energy-emitting tip proximal to a distal end of an elongated extension, the elongated extension and the energy-emitting tip having sizes and shapes that enable the energy-emitting tip to be inserted within the patient's vagina and to be located at substantially any depth within the vagina.

The method further includes locating the energy-emitting tip adjacent to external skin in the patient's pubic area, thereby heating and tightening the external skin, and inserting the energy-emitting tip into the patient's vagina, thereby heating and shrinking tissue within the vagina.

In embodiments, the wand is connected to the cosmetic device by an umbilical cable. In some embodiments, the skin-heating energy is laser energy. In other embodiments, the skin-heating energy is infrared light energy. In other embodiments, the skin-heating energy is heat that is transferred to the skin by direct thermal contact.

In various embodiments, the designers of the wand did not anticipate insertion thereof into a vagina. While in certain embodiments the designers of the wand anticipated insertion thereof into a vagina.

In some embodiments, the wand is kept in substantially constant motion while it is heating the external skin and while it is heating the tissue within the vagina. Other embodiments further include alternately repeating the steps of locating the energy-emitting tip adjacent to the external skin and inserting the energy-emitting tip into the vagina.

Various embodiments further include applying a lubricant within the vagina before insertion therein of the energy-emitting tip. And certain embodiments further include applying an energy-conducting substance to the exterior skin before locating the energy-emitting tip adjacent thereto.

In embodiments, the energy-conducting substance is ultrasound gel. Some embodiments further include waiting for a patient recovery time to elapse, and repeating the steps of locating and inserting.

Various embodiments further include installing a disposable cover over the distal end of the elongated extension before inserting the energy-emitting tip into the patient's vagina. And in some of these embodiments a distal end of the disposable cover is open, thereby exposing at least a portion of the energy-emitting tip.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### BRIEF DESCRIPTION OF THE DRAWINGS

FIG. 1 is a photograph of an RF-emitting cosmetic apparatus used in an embodiment of the present invention;

FIG. 2 is a close-up photograph of an RF-emitting probe used with the apparatus of FIG. 1;

FIG. 3 is a flow diagram illustrating an embodiment of the present invention;

FIG. 4 is a photograph showing the probe of FIG. 2 being applied to the external pubic area of a female patient;

FIG. 5 is a photograph showing the probe of FIG. 2 being applied to the inside of the vagina of a patient; and

FIG. 6 is a side view illustrating the placement of a protective cover over a probe in an embodiment of the present invention.

### DETAILED DESCRIPTION

The present invention is a non-surgical method whereby a single procedure is applied by a single, appropriately trained technician using a single cosmetic apparatus to improve both the external pubic appearance and the internal vaginal tightness of a female patient's body, thereby providing a more youthful appearance and heightened sexual pleasure without excessive costs, pain, or recovery time.

The method employs a device that is at least similar to an existing device that is typically used to tighten external skin in regions of the body such as the abdomen, thighs, love handles, face, and neck that are normally visible. However, in the present invention the device is used in a novel manner that was not foreseen by previous device manufacturers.

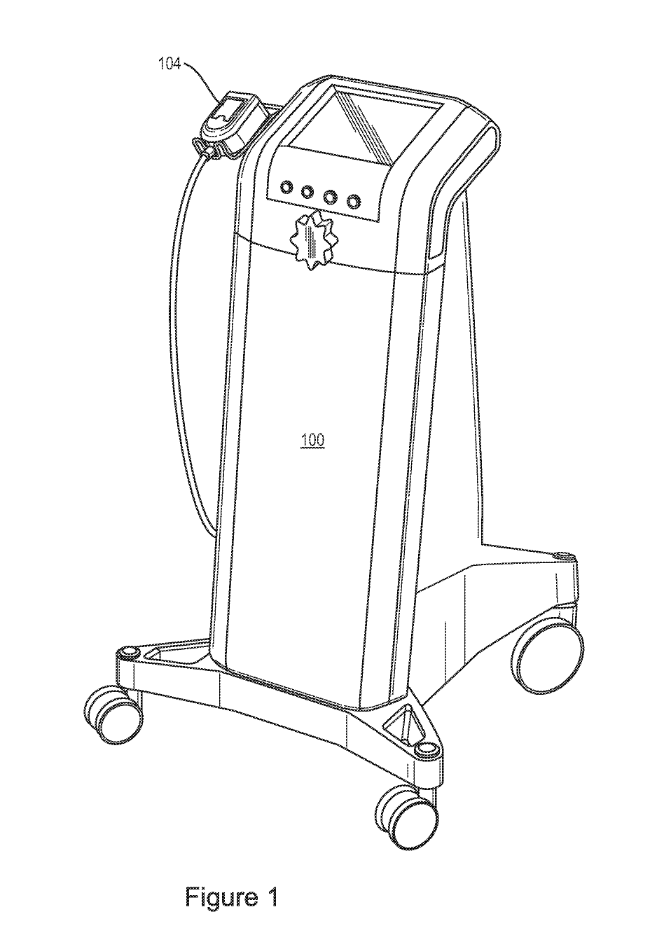

An example of a device used in some embodiments is illustrated in FIG. 1. The model shown in FIG. 1 includes a “wand” 104 connected by an umbilical cord to the main unit 100. The wand delivers RF heating energy to the skin, thereby heating collagen in the skin, and causing the collagen to contract and thicken, while also triggering the creation of new collagen, and thereby leading to tightening of the skin. In other embodiments, collagen heating and skin tightening is induced by direct thermal transfer, or by the application of infrared or laser energy to the skin.

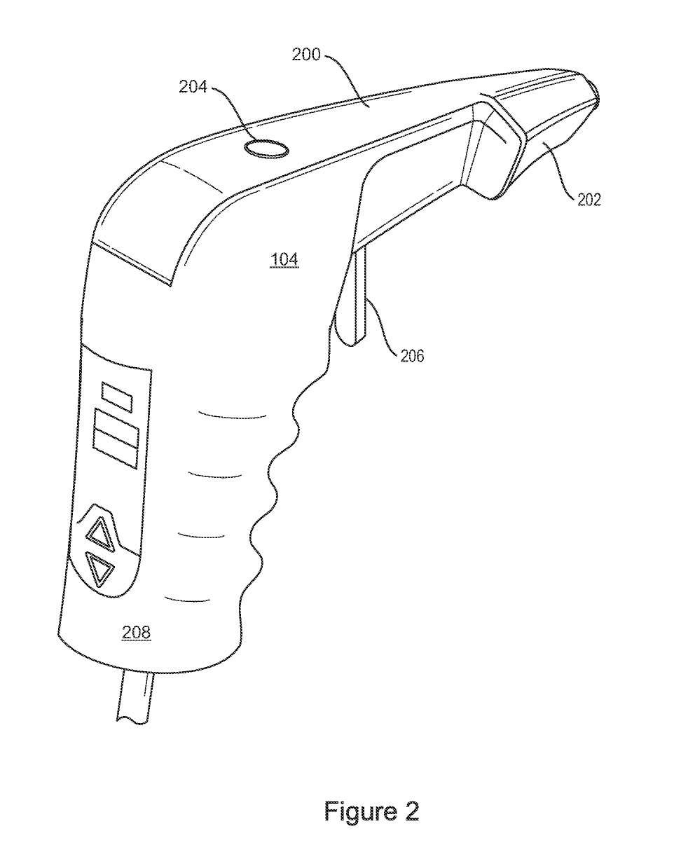

According to the method of the present invention, a cosmetic device 100 such as the device 100 shown in FIG. 1 includes a wand 104 having a size and shape that allow it to be inserted within the vagina. This enables the same device 100 to be used to heat tissue within the vaginal canal as well as in the external pubic area. FIG. 2 illustrates a wand 104 that is included with some embodiments. The probe 104 includes an elongated extension 200 with a tip 202 that has a shape and size suitable for insertion into the vagina of a patient. The heat output can be controlled either using a button 204 on top of the wand 104, or a trigger 206 provided in front of the grip handle 208.

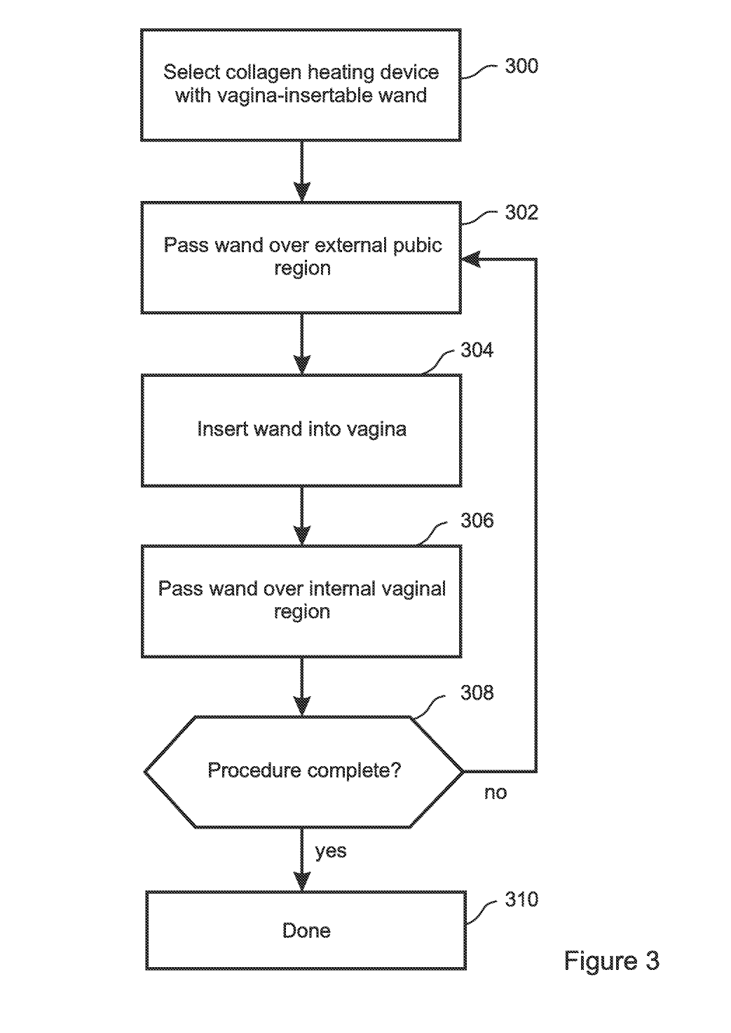

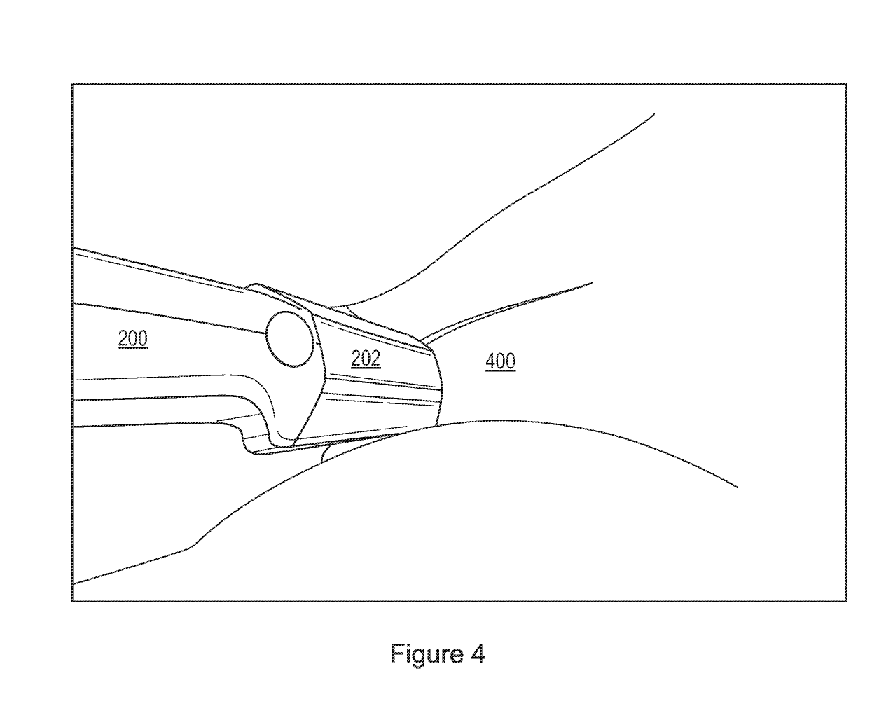

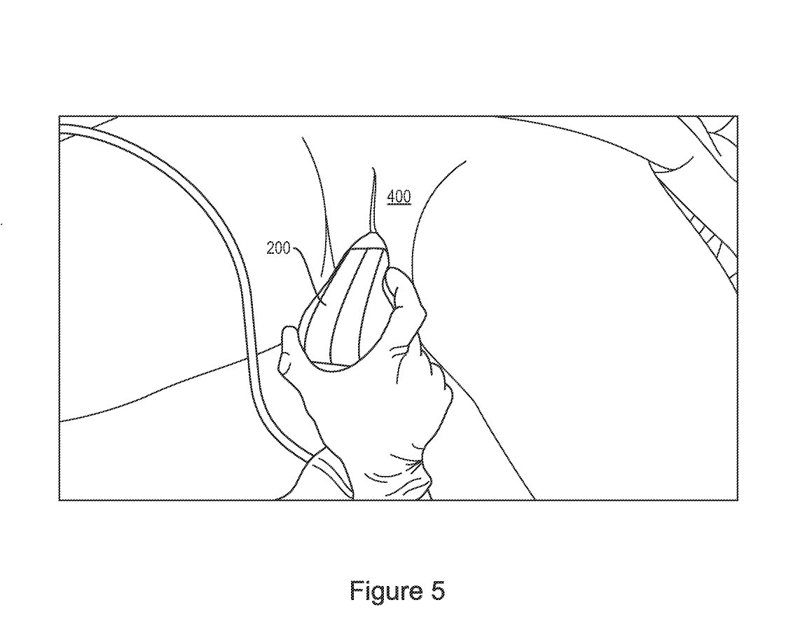

With reference to FIG. 3, according to the method of the present invention a collagen-heating device 100 is selected 300 which includes a wand 104 having a size and shape that allows the tip 202 of the wand to be inserted into the vagina. In some embodiments an energy conducting substance such as ultrasound gel is applied to the exterior pubic area, and a lubricant is applied within the vagina. With reference to FIGS. 3, 4, and 5, the tip 202 of the wand 104 is passed 302 over the external pubic area 400 (FIG. 4) and then inserted into the vagina 304 and passed over the tissues within the vagina 306 (FIG. 5). Application of the collagen-heating tip 104 alternately to the external pubic area 302 and the internal vaginal area 304 is repeated 308 until a desired degree of collagen heating has been effected in all of the treated areas 310.

By shifting the wand 104 frequently between the external pubic and internal vaginal regions, excessive overheating and discomfort in any one region is avoided. Typically, the patient feels a warming sensation, but does not experience any undue discomfort, so that anesthesia is not typically required.

Accordingly, during a single, non-surgical procedure both the external skin of the pubic area, as illustrated in FIG. 4, and the internal skin within the vagina, as illustrated in FIG. 5, are heated and/or irradiated, and are thereby tightened. The method can be repeated after a minimal recovery period, so as to provide a greater net effect. In some embodiment, the method can be repeated up to six times or more.

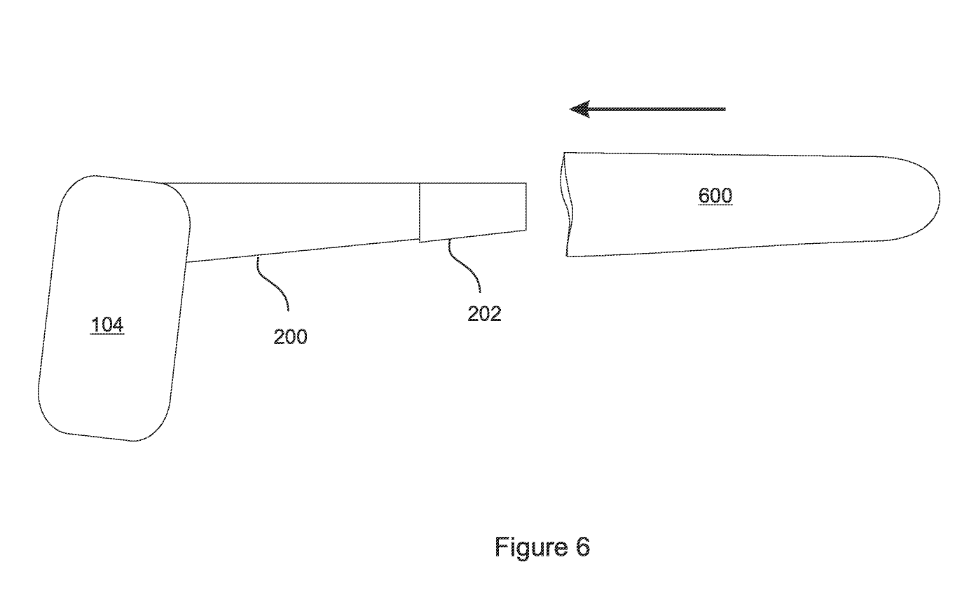

With reference to FIG. 6, in some embodiments, the extended portion 200, 202 of the wand 104 is covered during the procedure by a cleanable or disposable cover 600, so as to facilitate cleaning and sterilization of the wand 104 after use. In some embodiments, a finger of a surgical glove can be used to cover the wand, and in some embodiments the tip of the sleeve or glove finger is cut off so as to avoid interference with the heat application.

In various embodiments, the wand 104 emits radio frequency energy, laser energy, or conductive heat energy. In some embodiments, the wand 104 is commercially available from the manufacturer of the cosmetic apparatus 100, while in other embodiments a special wand attachment 104 is provided for use with an existing cosmetic apparatus 100.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A method applicable to a female patient for tightening tissue in both the external pubic area and the internal vaginal area during a single treatment session using a single device, the method comprising:
providing a cosmetic device that is able to emit energy from a wand that heats collagen in skin, the wand having an energy-emitting tip proximal to a distal end of an elongated extension, the elongated extension and the energy-emitting tip having sizes and shapes that enable the energy-emitting tip to be inserted within the patient's vagina and to be located at substantially any depth within the vagina;
locating the energy-emitting tip adjacent to external skin in the patient's pubic area, thereby heating and tightening the external skin; and
inserting the energy-emitting tip into the patient's vagina, thereby heating and shrinking tissue within the vagina.

2. The method of claim 1, wherein the wand is connected to the cosmetic device by an umbilical cable.

3. The method of claim 1, wherein the skin-heating energy is laser energy.

4. The method of claim 1, wherein the skin-heating energy is infrared light energy;

5. The method of claim 1, wherein the skin-heating energy is heat that is transferred to the skin by direct thermal contact.

6. The method of claim 1, wherein designers of the wand did not anticipate insertion thereof into a vagina.

7. The method of claim 1, wherein designers of the wand anticipated insertion thereof into a vagina.

8. The method of claim 1, wherein the wand is kept in substantially constant motion while it is heating the external skin and while it is heating the tissue within the vagina.

9. The method of claim 1, further comprising alternately repeating the steps of locating the energy-emitting tip adjacent to the external skin and inserting the energy-emitting tip into the vagina.

10. The method of claim 1, further comprising applying a lubricant within the vagina before insertion therein of the energy-emitting tip.

11. The method of claim 1, further comprising applying an energy-conducting substance to the exterior skin before locating the energy-emitting tip adjacent thereto.

12. The method of claim 11, wherein the energy-conducting substance is ultrasound gel.

13. The method of claim 1, further comprising:
waiting for a patient recovery time to elapse; and
repeating the steps of locating and inserting.

14. The method of claim 1, further comprising installing a disposable cover over the distal end of the elongated extension before inserting the energy-emitting tip into the patient's vagina.
